(19) United States
(12) Patent Application Publication
(10) Pub. No.: US 2021/0052349 A1
(43) Pub. Date: Feb. 25, 2021

(54) ROBOTIC OPERATING TABLE

(71) Applicant: MEDICAROID CORPORATION, Kobe-shi (JP)

(72) Inventors: Yutaro YANO, Kobe-shi (JP); Mitsuichi HIRATSUKA, Kobe-shi (JP); Hiroki ONO, Kobe-shi (JP)

(73) Assignee: MEDICAROID CORPORATION, Kobe-shi (JP)

(21) Appl. No.: 16/996,896

(22) Filed: Aug. 19, 2020

(30) Foreign Application Priority Data
Aug. 23, 2019 (JP) 2019-152574
Jun. 5, 2020 (JP) 2020-098496

(51) Int. Cl. A61B 90/50; A61B 90/25; A61B 90/00; A61B 34/30; A61B 34/20; A61B 5/055; A61B 5/06; G16H 40/67

(52) U.S. Cl. CPC A61B 90/50; A61B 90/25; A61B 90/37; A61B 90/39; A61B 2034/2068; A61B 34/20; A61B 5/0555; A61B 5/06; G16H 40/67; A61B 34/30

(57) ABSTRACT

A robotic operating table according to an embodiment may include: a tabletop on which a patient can be placed; an articulated robot arm that supports the tabletop to be movable; and a communication part to communicate with the inspection information management device. The communication part is configured to receive, from the inspection information management device, attention position information indicating an attention position in the patient specified based on an image of the patient captured by the medical imaging device. The articulated robot arm is configured, based on the attention position information received from the inspection information management device, to move the tabletop to a surgery-performing tabletop position which is the position of the tabletop at the time when surgery is executed on the patient.

### CROSS REFERENCE TO RELATED APPLICATIONS

This application claims priority based on 35 USC 119 from prior Japanese Patent Application No. 2019-152574 filed on Aug. 23, 2019 and prior Japanese Patent Application No. 2020-098496 filed on Jun. 5, 2020, the entire contents of all of which are incorporated herein by reference.

### BACKGROUND

The disclosure may relate to a robotic operating table, and more particularly to a robotic operating table that moves a tabletop by an articulated robot arm.

In a related art, a robotic operating table in which a tabletop is moved by an articulated robot arm is known (for example, Patent Document 1).

Patent Document 1 discloses a robotic bed (robotic operating table) including a tabletop on which a patient can be placed and an articulated robot arm that supports the tabletop. The robotic bed is configured so that the patient placed on the table can be moved by moving the tabletop by the articulated robot arm. Specifically, Patent Document 1 discloses the robotic bed that is configured to move the patient between an imaging position where the patient is imaged by an MRI (magnetic resonance imaging) device and an operative field where a medical staff such as a doctor treats the patient. Thus, the robotic bed is capable of smoothly moving the patient when imaging the patient with the MRI device and performing treatment or surgery on the patient.

Patent Document 1: Domestic re-publication of PCT international application No. 2017/098543

### SUMMARY

However, the robotic bed disclosed in Patent Document 1 may still have a room for improving convenience when imaging a patient by a medical imaging device such as an MRI device and performing treatment or surgery on the patient.

An object of an embodiment of the disclosure may be to provide a robotic operating table capable of improving convenience when imaging a patient by a medical imaging device and performing treatment or surgery on the patient.

A first aspect of the disclosure may be a robotic operating table communicable with an inspection information management device via a network. The robotic operating table may include: a tabletop on which a patient can be placed; an articulated robot arm that supports the tabletop to be movable; and a communication part to communicate with the inspection information management device. The communication part is configured to receive, from the inspection information management device, attention position information indicating an attention position in the patient specified based on an image of the patient captured by a medical imaging device. The articulated robot arm is configured, based on the attention position information received from the inspection information management device, to move the tabletop to a surgery-performing tabletop position which is the position of the tabletop at the time when surgery is executed on the patient.

According to the first aspect, the surgery can be started on the attention position in the patient placed on the tabletop, only by moving the tabletop to the surgery-performing tabletop position. Therefore, the first aspect can provide the robotic operating table capable of improving user convenience upon imaging the patient by the medical imaging device and performing treatment or surgery on the patient. Note that in this disclosure, supporting a tabletop means a broad concept that includes supporting the tabletop directly or indirectly.

A second aspect of the disclosure may be a robotic operating table communicable with an inspection information management device via a network. The robotic operating table may include: a tabletop on which a patient can be placed; an articulated robot arm that supports the tabletop to be movable; and a communication part to communicate with the inspection information management device. The communication part is configured to receive, from the inspection information management device, attention position information indicating an attention position in the patient specified based on an image of the patient captured by a medical imaging device. The articulated robot arm is configured to rotate the tabletop about the attention position in the patient on the tabletop based on the attention position information received from the inspection information management device.

According to the second aspect, the patient placed on the tabletop can be rotated about the attention position in the patient on the tabletop. Thus, it is possible to change an angle of viewing (observation angle) the attention position from the medical staff in a state where the attention position is fixed. That is, the medical staff can observe the attention position in the patient from various angles without moving the medical staff himself or herself. Therefore, the second aspect can provide the robotic operating table capable of improving user convenience upon imaging the patient by the medical imaging device and performing treatment or surgery on the patient.

According to at least one of the above described aspects, it may be possible to improve user convenience when imaging the patient by the medical imaging device and performing treatment or surgery on the patient.

### BRIEF DESCRIPTION OF DRAWINGS

FIG. 1 is a diagram illustrating a perspective view of a robotic operating table according to a first embodiment;

FIG. 2 is a diagram illustrating a schematic cross-sectional view illustrating a tabletop, a tabletop holder, and a tabletop slide mechanism of the robotic operation table according to a first embodiment;

FIG. 3 is a diagram illustrating a plan view of the robotic operating table according to a first embodiment;

FIG. 4 is a block diagram illustrating a control configuration of the robotic operating table according to a first embodiment;

FIG. 5 is a diagram illustrating a perspective view of an operation device including a joystick for a robotic operating table according to a first embodiment;

FIG. 6 is a diagram illustrating a perspective view of an operation device including a move direction receiver for a robotic operating table according to a first embodiment;

FIG. 7 is a diagram for explaining an imaging preparation position of the robotic operating table according to a first embodiment;

FIG. 8 is a diagram for explaining an imaging position of the robotic operating table according to a first embodiment.

FIG. 9 is a diagram for explaining a surgery-performing tabletop position of the robotic operating table according to a first embodiment;

FIG. 10A is a diagram for explaining a yaw rotation as an example of rotation of the robotic operating table about an attention position according to a first embodiment;

FIG. 10B is a diagram for explaining a pitch rotation as an example of rotation of the robotic operating table about the attention position according to a first embodiment;

FIG. 10C is a diagram for explaining a roll rotation as an example of rotation of the robotic operating table about the attention position according to a first embodiment.

FIG. 11 is a diagram illustrating a perspective view of a robotic operating table and a surgical navigation system according to a second embodiment.

FIG. 12 is a block diagram illustrating a control configuration of the robotic operating table according to a second embodiment;

FIG. 13 is a diagram for explaining position recognition markers of the surgical navigation system according to a second embodiment.

### DETAILED DESCRIPTION

Descriptions are provided hereinbelow for one or more embodiments based on the drawings. In the respective drawings referenced herein, the same constituents are designated by the same reference numerals and duplicate explanation concerning the same constituents is omitted. All of the drawings are provided to illustrate the respective examples only.

### First Embodiment

### (Configuration of Robotic Operating Table)

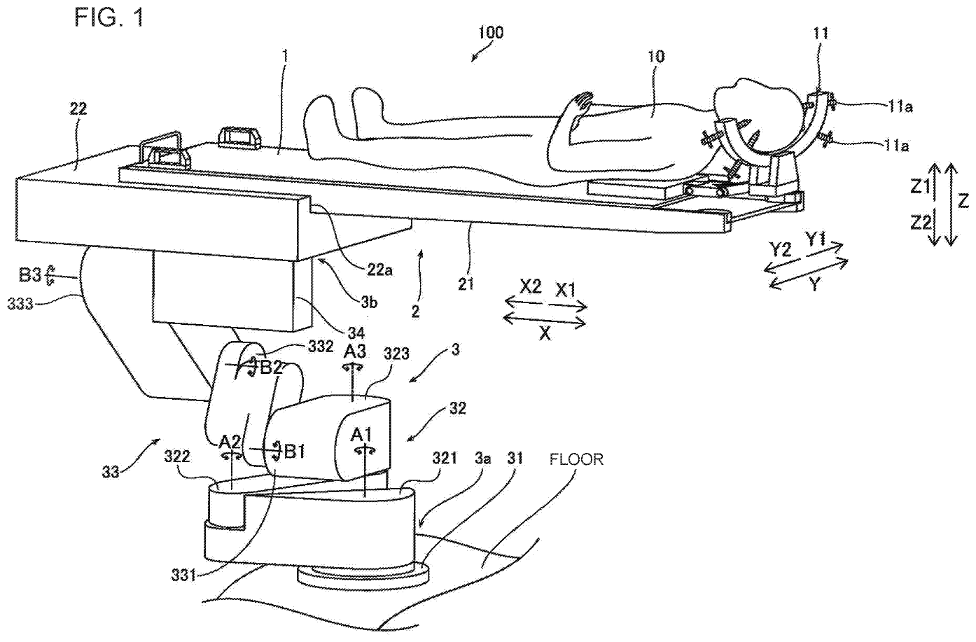

A configuration of a robotic operating table 100 according to a first embodiment is explained with reference to FIG. 1 to FIG. 4.

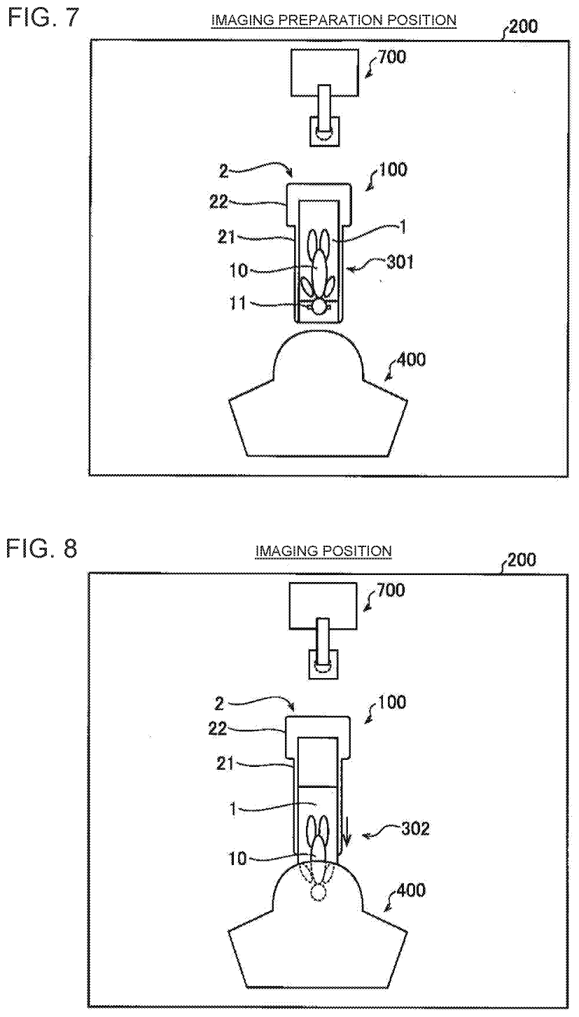

As illustrated in FIG. 1, the robotic operating table 100 is used as an operating table for operations performed in a setting such as a surgery or internal medicine setting. The robotic operating table 100 is used in a state of being arranged in the operating room 200 (see FIGS. 7-9). The robotic operating table 100 is capable of moving a tabletop 1 (or a top plate) to a loading position for loading the patient 10 onto the tabletop 1. Further, the robot operation table 100 is configured to move the patient 10 to desired positions (for example, an imaging preparation position 301, an imaging position 302 (image-capturing tabletop position), a surgery-performing tabletop position 303, etc.) by moving the tabletop 1 in a state where the patient 10 is placed on the tabletop 1. Also, the robotic operating table 100 is configured to tilt the patient 10 by tilting the tabletop 1 in the state where the patient 10 is placed on the tabletop 1.

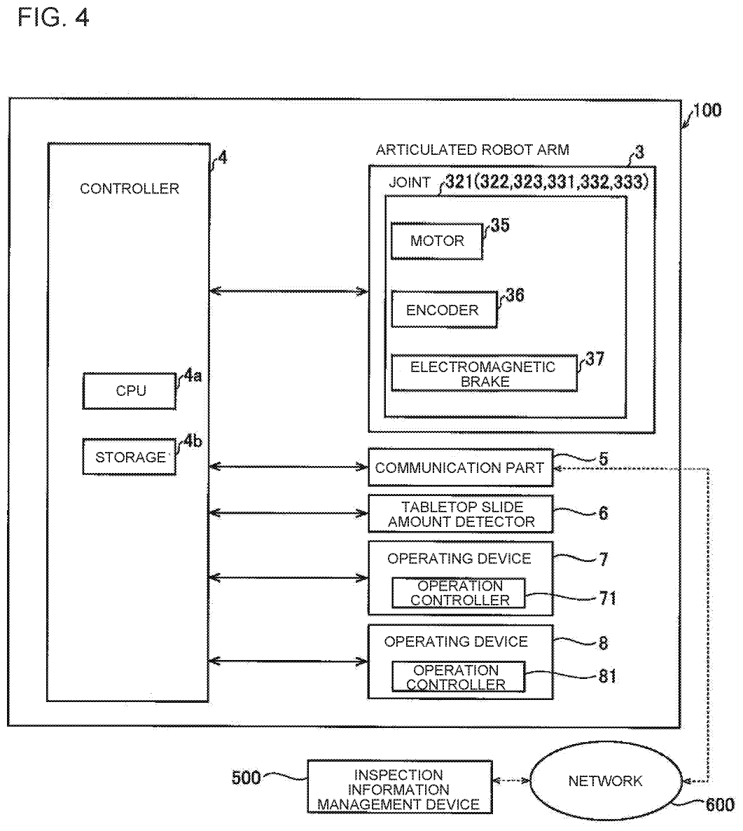

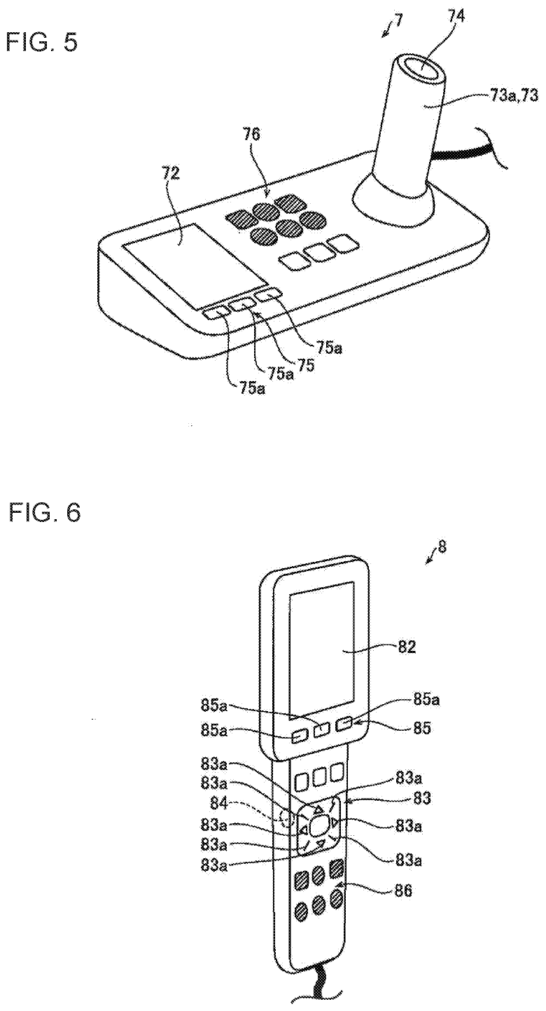

The robotic operating table 100 includes the tabletop 1 on which a patient is to be placed, a tabletop holder 2, an articulated robot arm 3, a controller 4 (see FIG. 4), a communication part 5 (see FIG. 4), a tabletop slide amount detector 6 (see FIG. 4), an operating device 7 (see FIG. 4 and FIG. 5), and an operating device 8 (see FIGS. 4 and 6).

The tabletop 1 is formed in a substantially rectangular shape. The upper surface of the tabletop 1 is formed to be substantially flat. The patient 10 is to be placed on the upper surface of the tabletop 1. Note that, the tabletop 1 is rotatable about an axis extending in the vertical direction (Z direction). In this disclosure, the horizontal direction along the longitudinal direction of the tabletop 1 is referred to as the X direction and the horizontal direction along the transverse direction of the tabletop 1 is referred to as the Y direction. In other words, the X direction and the Y direction represent directions based on the tabletop 1. The tabletop 1 is formed of, for example, a non-magnetic material such as glass fiber reinforced plastic (GFRP) or a resin material such as carbon fiber reinforced plastic (CFRP). In this way, an image of the patient 10 can be captured by using nuclear magnetic resonance, X rays, or the like in the state where the patient 10 is placed on the tabletop 1.

The tabletop 1 is provided with a head holder 11 to hold the head of the patient 10. The head holder 11 is provided at an end portion in the X1 side of the tabletop 1. Further, the head holder 11 is configured to fix the head of the patient 10 placed on the tabletop 1. Specifically, the head holder 11 includes plural head pins 11a as fixtures. That is, the head holder 11 is configured to fix the head of the patient 10 placed on the tabletop 1, with plural head pins 11a of the head holder 11. The head holder 11 is formed of, for example, a resin material such as polyetheretherketone (PEEK), polyamideimide (PAI), carbon fiber reinforced plastic (CFRP), or a non-magnetic material such as titanium. In this way, an image of the patient 10 can be captured by using nuclear magnetic resonance, X rays, or the like in the state where the head of the patient 10 is held by the head holder 11.

The tabletop 1 is configured to be movable by the articulated robot arm 3. Specifically, the tabletop 1 is movable in the X direction, which is a horizontal direction, in the Y direction, which is a horizontal direction perpendicular to the X direction, and in the Z direction, which is the vertical direction perpendicular to the X direction and the Y direction. Moreover, the tabletop 1 is rotatable (capable of being caused to roll) about an axis extending in the X direction. The tabletop 1 is also rotatable (capable of being caused to pitch) about an axis extending in the Y direction. The tabletop 1 is also rotatable (capable of being caused to yaw) about an axis extending in the Z direction. Further, the tabletop 1 is configured to be moved in the X direction by the tabletop slide mechanism 23.

The tabletop holder 2 is configured to hold the tabletop 1. Specifically, the tabletop holder 2 includes a first holding part 21 that holds the tabletop 1 to be slidably movable with respect to the first holding part 21, and a second holding part 22 (top frame) that holds the first holding part 21. The second holding part 22 is connected to the articulated robot arm 3. The first holding part 21 is connected to a recess 22a of the second holding part 22 and projects from the second holding part 22 toward the X1 side.

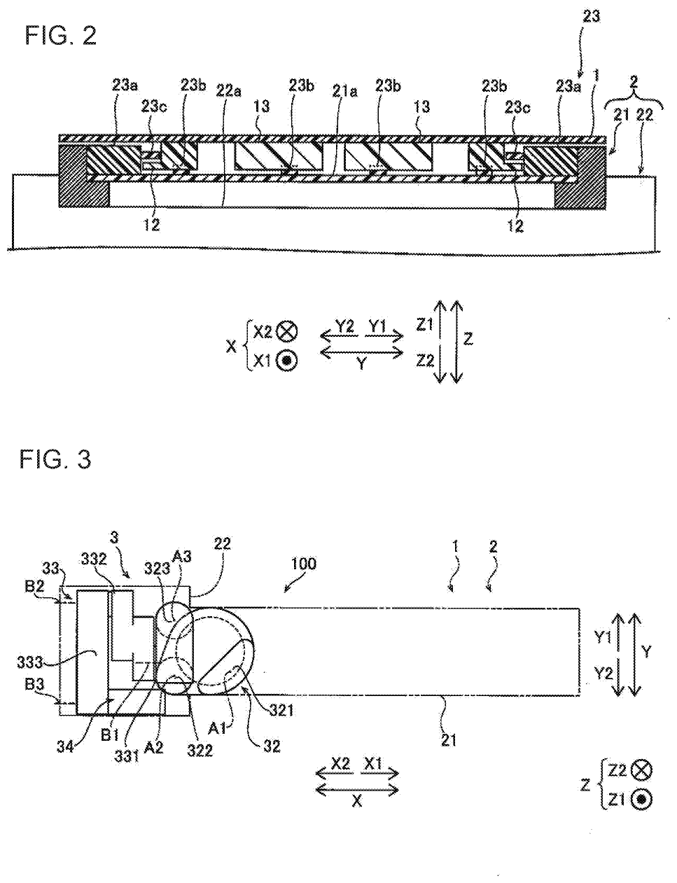

As illustrated in FIG. 2, the robotic operating table 100 includes a tabletop slide mechanism 23 (see FIG. 2). The tabletop slide mechanism 23 includes guides 23a and plural rollers 23b. The guides 23a are configured to support the tabletop 1 to be slidable in the X direction. The guides 23a are configured to guide guided portions 12 of the tabletop 1. The guides 23a are provided on both sides of the first holding part 21 of the tabletop holder 2 in the Y direction. The plural rollers 23b are arranged on an upper surface of a mounting member 21a of the first holding part 21 of the tabletop holder 2 so that the tabletop 1 can be slid in the X direction. The plural rollers 23b are provided in roller holding portions 13 of the tabletop 1 and the guided portions 12 which also serve as roller holding portions. The guides 23a are provided with a plurality of disengagement prevention portions 23c. The disengagement prevention portions 23c are configured to prevent the tabletop 1 from rising in the Z direction and prevent the tabletop 1 from coming off the first holding part 21 when the tabletop 1 is slid in the X direction. The disengagement prevention portions 23c are projected from the guides 23a in the Y direction and opposed to the guided portions 12 of the tabletop 1 in the Z direction. The disengagement prevention portions 23c are interfered with the guided portions 12 of the tabletop 1 so as to prevent the tabletop 1 from floating away in the Z direction.

The first holding part 21 of the tabletop holder 2 includes a lock mechanism (not illustrated) to lock the tabletop 1. With this lock mechanism, the tabletop 1 is configured so as not to move (not to slide) during surgery or the like or when the robotic operating table 100 is being moved. The lock mechanism includes a claw(s) to be engageable with the tabletop 1 and is configured to lock (fix) the tabletop 1 to the first holding part 21 of the tabletop holder 2 by the claw(s). By releasing the lock of the tabletop by the lock mechanism, the tabletop 1 is movable in the X direction between a position (the position illustrated in FIG. 1) where the entire tabletop 1 is held by the first holding part 21 of the tabletop holder 2 and a position (the position illustrated in FIG. 8) where a distal end portion of the tabletop 1 projects from the first holding part 21 of the tabletop holder 2 toward the X1 side. The distal end portion of the tabletop 1 that is projected from the first holding part 21 of the tabletop holder 2 is to be inserted in a medical imaging device 400 (see FIGS. 7 to 9). Note that the tabletop slide mechanism 23 and the lock mechanism of the tabletop 1 may be driven manually or be driven by a driver such as a motor or the like.

As illustrated in FIG. 1, the articulated robot arm 3 is configured to support the tabletop 1 to be movable. The articulated robot arm 3 is configured to move the tabletop 1. The articulated robot arm 3 includes a first end portion 3a supported by a base 31 fixed to the floor and a second end portion 3b (an end of a later-described pitch rotation mechanism 34) that supports the tabletop 1. The first end portion 3a of the articulated robot arm 3 is supported on the base 31 such that the first end portion 3a of the articulated robot arm 3 is rotatable about a base rotation axis (rotation axis A1) extending in the vertical direction (Z direction). The base 31 is provided at a substantially center of a movement range of the tabletop 1 in a plan view (as seen in the Z direction). The second end portion 3b of the articulated robot arm 3 is configured to support the tabletop 1 via the second holding part 22 of the tabletop holder 2. That is, the articulated robot arm 3 supports the tabletop 1 via the second holding part 22 of the tabletop holder 2, and thus can move the tabletop holder 2 and the tabletop 1 together. The second end portion 3b of the articulated robot arm 3 is configured to support the second holding part 22 provided at one end in the longitudinal direction of the tabletop holder 2.

As illustrated in FIG. 3, the articulated robot arm 3 is capable of being disposed in such a posture to be entirely hidden under the tabletop 1 (the Z2 side) in the plan view. For example, the articulated robot arm 3 is configured to be housed (retracted) in an accommodation space which is a space under the tabletop 1 and the second holding part 22 of the tabletop holder 2, when the tabletop 1 is positioned at a surgery-performing tabletop position. Specifically, the articulated robot arm 3 is capable of being disposed in such a posture as to be folded and completely hidden under the tabletop 1 and the second holding part 22 of the tabletop holder 2 in the plan view in a state where the articulated robot arm 3 has moved the tabletop 1 to a position for performing a surgical operation or treatment on the patient 10 placed on the tabletop 1. Meanwhile, the length of the articulated robot arm 3 in the folded posture in the direction parallel to the longitudinal direction of the tabletop 1 is equal to or shorter than ½ of the length of the tabletop 1 and the tabletop holder 2 in the longitudinal direction of the tabletop 1.

As illustrated in FIGS. 1 and 3, the articulated robot arm 3 includes a horizontal articulated assembly 32, a vertical articulated assembly 33, and a pitch rotation mechanism 34. The horizontal articulated assembly 32 includes horizontal joints 321, 322, and 323. The vertical articulated assembly 33 includes vertical joints 331, 332, and 333.

The articulated robot arm 3 is configured to move the tabletop 1 with seven degrees of freedom. Specifically, with the horizontal articulated assembly 32, the articulated robot arm 3 has three degrees of freedom to rotate about the rotation axis A1 extending in the vertical direction, rotate about a rotation axis A2 extending in the vertical direction, and rotate about a rotation axis A3 extending in the vertical direction. Further, with the vertical articulated assembly 33, the articulated robot arm 3 has three degrees of freedom to rotate about a rotation axis B1 extending the horizontal direction, rotate about a rotation axis B2 extending in the horizontal direction, and rotate about a rotation axis B3 extending in the horizontal direction. Furthermore, with the pitch rotation mechanism 34, the articulated robot arm 3 has one degree of freedom to allow the tabletop 1 to pitch about a rotation axis extending in the transverse direction of the tabletop 1 (Y direction). Note that the tabletop slide mechanism 23 has one degree of freedom to move the tabletop 1 in the X direction. Thus, the robotic operating table 100 is configured to move the tabletop 1 with eight degrees of freedom.

As illustrated in FIG. 4, each of the joints of the articulated robot arm 3 (the horizontal joints 321 to 323 and vertical joints 331 to 333) includes a motor 35, an encoder 36, an electromagnetic brake 37, and a reducer (not illustrated). The motor 35 is a drive source for the joint to rotate the tabletop 1. The motor 35 includes a servomotor. The motor 35 is driven based on control by the controller 4. The encoder 36 measures the amount of rotation of the motor 35. The encoder 36 transmits the result of the measurement of the amount of rotation of the motor 35 to the controller 4. The controller 4 is configured, based the measurement result of the encoder 26, to obtain information on the position of the tabletop 1. The electromagnetic brake 37 is a negative actuated-type electromagnetic brake that stops the rotation of the target driven by the motor 35. Specifically, the electromagnetic brake 37 cancels braking of the motor 35 when the motor 35 is energized, and brakes the motor 35 when the motor 35 is de-energized. The electromagnetic brake 37 may be an electromagnetic brake incorporated in the motor 35 or an electromagnetic brake externally mounted to the motor 35.

Also, the articulated robot arm 3 causes the tabletop 1 to yaw about an axis extending in the vertical direction (Z direction) by using at least one of the horizontal joints (at least one of 321, 322, and 323). Further, the articulated robot arm 3 causes the tabletop 1 to roll about an axis extending in the longitudinal direction (X direction) by using at least one of the vertical joints (at least one of 331, 332, and 333). Furthermore, the articulated robot arm 3 is configured to cause the tabletop 1 to pitch about an axis extending in the transverse direction (Y direction) by using the pitch rotation mechanism 34.

The controller 4 is control circuitry that may include, for example, a CPU (central processing unit) 4a, and a storage 4b such as a flash memory or the like. The controller 4 is disposed inside the base 31 and is configured to control movements of the tabletop 1 by the articulated robot arm 3. Specifically, the controller 4 moves the tabletop 1 by controlling the motion of the articulated robot arm 3 based on an operation input by a medical staff (user).

The communication part 5 is configured to communicate with an inspection information management device 500. The communication part 5 is communicably connected to the inspection information management device 500 via a network 600 such as LAN (local area network) in the hospital or the like. The inspection information management device 500 may be a host computer provided in the hospital, a control device of the medical imaging device 400, or the like, for example.

The tabletop slide amount detector 6 is configured to detect an amount of the slide movement of the tabletop 1 by the tabletop slide mechanism 23. For example, in a case where a wire is provided which moves in response to the slide movement of the tabletop 1 by the tabletop slide mechanism 23, the tabletop slide amount detector 6 may detect the slide movement amount of the wire as the slide movement amount of the tabletop 1. Further, in a case where the driver such as a motor is provided to slidably move the tabletop 1, the tabletop slide amount detector 6 may be configured as a position detection device of the driver such as an encoder, potentiometer, or the like. The tabletop slide amount detector 6 is configured to transmit to the controller 4 the detection result of the slide movement amount of the tabletop 1 by the tabletop slide mechanism 23.

As illustrated in FIG. 4 to FIG. 6, the operating device 7 and the operating device 8, serving as user interfaces, input devices, or manipulators, receive operations input by the medical staff (user) for moving the tabletop 1. The operating devices 7 and 8 are each capable of receiving operations for the robotic operating table 100. The operating device 7 is mainly used in a state where it is mounted to the tabletop holder 2. The operating device 8 is mainly used at a position away from the robotic operating table 100. The operating device 7 is detachably mounted to the second holding part 22 of the tabletop holder 2 with being engaged with engagement portions (not illustrated) provided on a side surface of the second holding part 22 of the tabletop holder 2. The operating devices 7 and 8 are connected to the controller 4 so as to be capable of making wired communication with the controller 4.

As illustrated in FIG. 4 and FIG. 5, the operating device 7 includes an operation controller 71, a display 72, a move operation reception part 73 as an input device, a trigger 74 as an input device, a registration operation reception part 75 as an input device, and a mode setting operation reception part 76 as an input device. As illustrated in FIG. 4 and FIG. 6, the operating device 8 includes an operation controller 81, a display 82, a move operation reception part 83 as an input device, a trigger 84 as an input device, a registration operation reception part 85 as an input device, and a mode setting operation reception part 86 as an input device.

The operation controller 71 (81) controls components in the operating device 7 (8) based on operations input by the medical staff (user). For example, the operation controller 71 (81) controls imagery to be displayed on the display 72 (82) based on an operation input by the medical staff (user). Moreover, the operation controller 71 (81) transmits operation (instruction) information indicating an operation input by the medical staff (user) to the controller 43. The controller 4 performs control that causes the articulated robot arm 3 to move the tabletop 1 based on the received operation information.

The display 72 (82) displays the state of the tabletop 1, the state of operation on the operating device 7 (8), an operation screen, and so on. The display 72 (82) includes a display, such as liquid crystal display or an organic EL (Electro Luminescence) display.

The move operation reception part 73 (83) receives a move operation (instruction) to move the tabletop 1 from the medical staff (user). The move operation reception part 73 of the operating device 7 includes a joystick 73a. The joystick 73a is operated by being tilted. The move operation reception part 83 of the operating device 8 includes multiple (eight) move direction receivers 83a provided respectively for directions in which to move the tabletop 1. In other words, the move direction receivers 83a are provided for eight directions. Each of the move direction receivers 83a receives an operation for moving the tabletop 1 by being pressed.

The trigger 74 (84) is provided to enable operations on the move operation reception part 73 (83). Specifically, the trigger 74 (84) has a function to allow energization of the motors 35 by being operated. The controller 4 performs control that energizes the motors 35 while the trigger 74 (84) is operated. Thus, operating the trigger 74 (84) cancels braking of the motors 35 by the electromagnetic brakes 37. Hence, only while the trigger 74 (84) is operated, operations on the move operation reception part 73 (83) are enabled and the tabletop 1 can be moved. Further, the trigger 74 (84) has a function to de-energize the motors 35 when the operation on the trigger 74 (84) is canceled. The controller 4 actuates the electromagnetic brakes 37 by stopping the energization of the motors 35 when the trigger 74 (84) is not operated. Thus, cancelling the operation on the trigger 74 (84) causes the electromagnetic brakes 37 to break the motors 35. Hence, when the trigger 74 (84) is not operated, any operations on the move operation reception part 73 (83) are disabled and the tabletop 1 cannot be moved.

The trigger 74 of the operating device 7 is provided at the tip of the joystick 73a. Pressing the trigger 74 of the operating device 7 enables operations on the joystick 73a. On the other hand, in the state where the pressing of the trigger 74 is canceled, any operations on the joystick 73a are disabled. The trigger 84 of the operating device 8 is provided at a surface of the operating device 8 opposite from a surface of the operation device where the move direction receivers 83a are provided. Pressing the trigger 84 of the operating device 8 enables operations on the move direction receivers 83a. On the other hand, in the state where the pressing of the trigger 84 is canceled, any operations on the move direction receivers 83a are disabled.

The registration operation reception part 75 (85) receives, from the user, a registration operation (instruction) to register a position of the tabletop 1 as a registered position and a set operation (instruction) to set the registered position as a movement destination of the tabletop 1. The registration operation reception part 75 (85) includes multiple (three) registration operation reception buttons 75a (85a). The registration operation reception part 75 (85) receives a registration operation and a set operation with each registration operation reception button 75a (85a). In other words, multiple different registered positions can be registered. The registration operation reception part 75 (85) receives a registration operation or a set operation when a registration operation reception button 75a (85a) is pressed.

When the registration operation reception part 75 (85) receives a registration operation, the controller 4 stores in the storage 4b the position of the tabletop 1 as the registered position. Also, when the registration operation reception part 75 (85) receives a set operation, the controller 4 sets the registered positions stored in the storage 4b as the movement destination of the tabletop 1. Then, when the move operation reception part 73 (83) receives a move operation with the registered position being set as the movement destination of the tabletop 1, the controller 4 controls the motion of the articulated robot arm 3 such that the tabletop 1 will be placed at the registered position stored in the storage 4b.

The mode setting operation reception part 76 (86) is configured to receive an operation to set a rotation mode from the user. The rotation modes are not limited; however, the rotation modes may include: a yaw mode in which the tabletop 1 is rotated in the horizontal plane about an axis extending in the vertical direction (Z direction); a pitch mode in which the tabletop 1 is rotated about an axis extending in a direction parallel to the traversing direction (Y direction) of the tabletop 1; and a roll mode in which the tabletop 1 is rotated about an axis extending in a direction parallel to the longitudinal direction (X direction) of the tabletop 1, for example. In the yaw mode, the tabletop 1 is caused to yaw based on an operation input by the medical staff (user). In the pitch mode, the tabletop 1 is caused to pitch based on an operation input by the medical staff (user). In the roll mode, the tabletop 1 is caused to roll based on an operation input by the medical staff (user). The tabletop 1 is moved when the trigger 74 (84) and the move operation reception part 73 (83) are operated in the state where one of the motion (rotation) modes selected by the mode setting operation reception part 76 (86).

### (Configuration Related to Intraoperative MRI Surgery)

Next, with reference to FIGS. 7 to 10, a configuration of the robotic operating table 100 related to an intraoperative magnetic resonance imaging (MRI) surgery. Note that the intraoperative MRI surgery means a surgical operation performed on the patient 10 in the operating room 200 while imaging the patient 10 with an MRI device (medical imaging device 400) arranged in the operating room 200. The intraoperative MRI surgery includes, for example, neurosurgery for removing a brain tumor. In the surgical operation for removing the brain tumor, confirmation of a tumor excision range, confirmation of remaining tumor, and the like are performed during the surgical operation based on a patient image obtained with the MRI device. Note that FIGS. 7 to 9 illustrate an example of performing neurosurgery to remove a brain tumor as an intraoperative MRI operation.

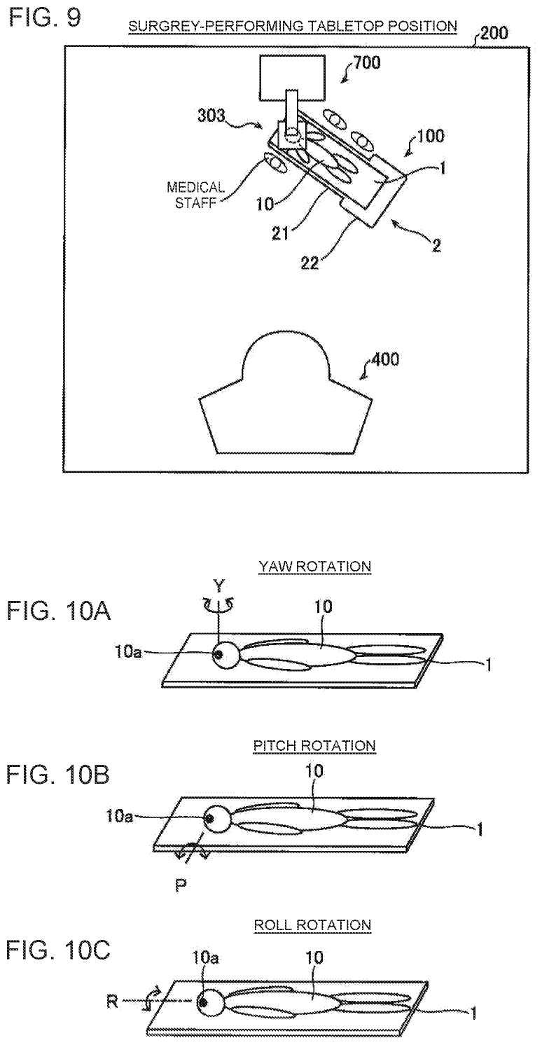

As illustrated in FIGS. 7 to 9, the articulated robot arm 3 of the robotic operating table 100 is configured to move the tabletop 1 among an imaging preparation position 301 (see FIG. 7) at which the tabletop 1 is ready to be insert into the medical imaging device 400, an imaging position 302 (see FIG. 8) at which an image of the patient 10 on the tabletop 1 is captured with the medical imaging device 400, and a surgery-performing tabletop position 303 (see FIG. 9) at which surgery is performed on the patient 10. Accordingly, the movement of the patient 10 (tabletop 1) to the imaging preparation position 301, to the imaging position 302 (image-capturing tabletop position 302), and to the surgery-performing tabletop position 303 can be performed quickly and accurately. In a first embodiment, the surgery-performing tabletop position 303 is the position of the tabletop 1 where the medical staff performs the surgical operation while observing the tumor in the head of the patient 10 with the surgical microscope 700 disposed in the operating room 200. Note that in FIGS. 7 to 9, illustration of the articulated robot arm 3 is omitted for convenience.

Specifically, the robotic operating table 100 is configured to move the tabletop 1 and the tabletop holder 2 between the surgery-performing tabletop position 303 and the imaging preparation position 301 by at least rotating the tabletop 1 and the tabletop holder 2 with the articulated robot arm 3. The imaging preparation position 301 is the position where the tabletop 1 is positioned close to the medical imaging device 400. In the state where the tabletop 1 is located at the imaging preparation position 301, there is a gap (distance) between the tabletop 1 and the medical imaging device 400. Thus, the tabletop 1 located at the imaging preparation position 301 is not in contact with the medical imaging device 400.

Further, the robotic operating table 100 is configured to slide the tabletop 1 to the imaging position 302 with the tabletop slide mechanism 23 after the tabletop 1 and the tabletop holder 2 are moved to the imaging preparation position 301 with the articulated robot arm 3. This configuration can make the range of movement of the tabletop 1 by the articulated robot arm 3 smaller than in a case where the tabletop 1 is moved to the imaging position 302 only by the articulated robot arm 3. Thus, the articulated robot arm 3 can be downsized. In the state where the tabletop 1 is located at the imaging position 302, a distal end portion of the tabletop 1 and the head (affected part) of the patient 10, which is a target to be imaged, have been inserted in the gantry which is the imaging part of the medical imaging device 400.

The medical staff (surgeon, assistant, etc.) can register, using the registration operation reception part 75 (85) of the operating device 7 (8), the imaging preparation position 301 and the surgery-performing tabletop position 303, as registered portions in advance. Further, in the case where the tabletop slide mechanism 23 is configured to slide the tabletop 1 by a driver such as a motor, the imaging position 302 where the head of the patient 10 is inserted into the gantry of the medical imaging device 400 can also be registered as a registered position in advance. In these cases, the movement of the patient 10 (tabletop 1) to the imaging preparation position 301, to the imaging position 302, and to the surgery-performing tabletop position 303 can be performed more quickly and accurately.

Here, in a first embodiment, the communication part 5 of the robotic operating table 100 is configured to receive attention position information indicating an attention position 10a (see FIGS. 10A to 10C) in the patient 10 specified based on an image of the patient captured by the medical imaging device 400. Further, the articulated robot arm 3 is configured to rotate the tabletop 1 about the attention position 10a based on the attention position information received from the inspection information management device 500. Thus, when imaging the patient 10 by the medical imaging device 400 and performing the treatment or surgery on the patient 10, the patient 10 placed on the tabletop 1 can be rotated about the attention position 10a of the patient 10 on the tabletop 1. As a result, it is possible to change the angle (observation angle) of the attention position 10a viewed from the medical staff in the state where the attention position 10a of the patient 10 on the tabletop 1 is fixed as the fixed position. Thus, the medical staff can observe the attention position 10a from various angles without moving the medical staff himself or herself. As a result, it is possible to improve the user convenience when imaging the patient 10 by the medical imaging device 400 and performing treatment or surgery on the patient 10.

The rotations of the tabletop 1 (the patient 10) about the attention position 10a may be carried out as illustrated in FIGS. 10A to 10C, for example; however, is not particularly limited. In the example illustrated in FIG. 10A, the articulated robot arm 3 rotates (yaw rotation) the tabletop 1 about the rotation axis Y extending through the attention position 10a in the direction perpendicular to the upper surface of the tabletop 1. In the example illustrated in FIG. 10B, the articulated robot arm 3 rotates (pitch rotation) the tabletop 1 about the rotation axis P extending through the attention position 10a in the direction parallel to the traversing direction of the tabletop 1. In the example illustrated in FIG. 10C, the articulated robot arm 3 rotates (roll rotation) the tabletop 1 about the rotation axis R extending through the attention position 10a in the direction parallel to the longitudinal direction of the tabletop 1.

The medical staff can set an attention position rotation mode for rotating the tabletop 1 about the attention position 10a by using the mode setting operation reception part 76 (86) of the operating device 7 (8). In a state where the attention position rotation mode is set by receiving the setting operation of the attention position rotation mode through the mode setting operation reception part 76 (86), the articulated robot arm 3 is configured, during the time when the move operation reception part 73 (83) receives the instruction to move the tabletop 1, to rotate the tabletop 1 about the attention position 10a. That is, only while the move operation reception part 73 (83) receives the instruction to move the tabletop 1, the tabletop 1 is rotated about the attention position 10a.

In a first embodiment, the attention position 10a is the position of an operative field (a field of view of a surgical site) in the patient 10. In this case, the attention position information is operative field position information indicating an operative field position in the patient 10. Thus, the patient 10 placed on the tabletop 1 can be rotated about the operative field position, which is a site of interest in surgery. Thus, it is possible to effectively improve the user convenience during surgery. Specifically, the operative field position is the position of the tumor in the patient 10 (the position of the brain tumor). In this case, the operative field position information is a tumor position information indicating the position of the tumor in the patient 10. Thus, the patient 10 placed on the tabletop 1 can be rotated around the tumor position which is the treatment site in the surgery. Thus, it is possible to effectively improve the user convenience during the surgery.

The articulated robot arm 3 is configured, based on the tumor position information, to rotate the tabletop 1 around the tumor position. Thus, when performing surgery on the patient 10 while observing the patient 10 by the surgical microscope 700, it is possible to rotate the tabletop 1 about the tumor position, which is a target position to be observed by the surgical microscope 700. Thus, the medical staff can observe the tumor position from various angles through the surgical microscope 700 without moving the surgical microscope 700.

Further, in a first embodiment, the controller 4 is configured to perform control for determining the tumor position in the patient 10 on the tabletop 1 with respect to the tabletop 1, based on the tumor position information obtained from the medical imaging device 400 and image-capturing tabletop position information indicating the image-capturing tabletop position which is the position of the tabletop 1 when imaging the patient 10 by the medical imaging device 400. Thus, it is possible to easily and accurately determine the tumor position in the patient 10 on the tabletop 1 (the on-tabletop tumor position, or the on-tabletop attention position). Further, the controller 4 is configured to perform control for determining the tumor position of the patient 10 on the tabletop 1 at the time of the surgery, based on on-tabletop tumor position information (on-tabletop attention position information) indicating the on-tabletop tumor position (the on-tabletop tumor position), and the surgery-performing tabletop position information indicating the position of the tabletop 1 at the time of the surgery on the patient 10. Thus, it is possible to easily and accurately reproduce the tumor position of the patient 10 on the tabletop 1 at the time of the surgery many times.

Specifically, the controller 4 determines the tumor position on the tabletop 1 (the on-tabletop tumor position), by the following equation (1).

P_{BED}=B_{IM}⁻¹×T×P_{IM} (1)

P_{BED}: a matrix indicating the tumor position with respect to the tabletop 1 (the on-tabletop tumor position)

B_{IM}: a matrix indicating the position of the tabletop 1 at the time of imaging in the coordinate system of the robotic operating table 100 (an example of the image-capturing tabletop position information)

T: a matrix indicating the distance between the coordinate system of the robot operation table 100 and the coordinate system of the medical imaging device 400

P_{IM}: a matrix indicating the tumor position at the time of imaging in the coordinate system of medical imaging device 400 (an example of the tumor position information)

The matrix B_{IM} of the equation (1) is expressed by the following equation (2).

B_{IM}=S_{IM}×b_{IM} (2)

S_{IM}: a matrix indicating the slide position (i.e., the position after the slide) of the tabletop 1 by the tabletop slide mechanism 23 at the time of the image-capturing in the coordinate system of the robotic operating table 100

b_{IM}: a matrix indicating the moved position of the tabletop 1 by the articulated robot arm 3 at the time of the image-capturing in the coordinate system of the robotic operating table 100

The matrix S_{IM} of the equation (2) can be determined in advance based on the output of the tabletop slide amount detector 6. The matrix b_{IM} of the equation (2) can be obtained based on the output of the encoder 36.

The matrix T of the equation (1) can be obtained based on the installation positions of the robot operation table 100 and the medical imaging device 400 in the operating room 200. The robotic operating table 100 can store the matrix T in advance in the storage 4b. The matrix P_{IM} of equation (1) can be obtained based on the image of the patient 10 captured by the medical imaging device 400. The robotic operating table 100 can obtain, via the communication part 5, the matrix P_{IM} obtained by the medical imaging device 400.

Further, the controller 4 determines, by using the following equation (3), the tumor position on the tabletop 1 at the time when the tabletop 1 is located at the surgery-performing tabletop position 303.

P_{SG}=B_{SG}×P_{BED} (3)

P_{SG}: a matrix indicating the tumor position on the tabletop 1 at the time when the tabletop 1 is located at the surgery-performing tabletop position 303

B_{SG}: a matrix indicating the position of the tabletop 1 at the time of surgery in the coordinate system of the robotic operating table 100 (an example of the surgery-performing tabletop position information)

P_{BED}: a matrix indicating the tumor position on the tabletop 1 (an example of the on-tabletop tumor position)

The matrix B_{SG} of the equation (3) is expressed by the following equation (4):

B_{SG}=S_{SG}×b_{SG} (4).

S_{SG}: a matrix indicating the slide position (i.e., the position before the slide) of the tabletop 1 by the tabletop slide mechanism 23 at the time of surgery in the coordinate system of the robotic operating table 100

b_{SG}: a matrix indicating the moved position of the tabletop 1 by the articulated robot arm 3 at the time of surgery in the coordinate system of the robotic operating table 100

The matrix S_{SG} of the equation (4) can be obtained based on the output of the tabletop slide amount detector 6. The matrix b_{SG} of the equation (4) can be obtained based on the output of the encoder 36.

The controller 4 controls the articulated robot arm 3 based on the matrix P_{SG} of the equation (3), so that the tabletop 1 rotates about the tumor position during the surgery.

Further, in a first embodiment, the articulated robot arm 3 is configured, based on the attention position information received from the inspection information management device 500, to move the tabletop 1 to match the attention position 10a with the surgical operation position to which the medical staff performs surgery while observing the patient 10 with the surgical microscope 700 disposed in the operating room 200. Thus, the attention position 10a of the patient 10 can be easily set to the surgical operation position, upon imaging the patient 10 by the medical imaging device 400 and performing the treatment or surgery on the patient 10. Thus, when imaging the patient 10 by the medical imaging device 400 and performing the treatment or surgery on the patient 10, the surgical operation on the attention position 10a of the patient 10 placed on the tabletop 1 can be started only by moving the patient 10 (the tabletop 1) to the surgery-performing tabletop position. Therefore, the user convenience can be improved. The articulated robot arm 3 is configured to move the tabletop 1 to the surgery-performing tabletop position 303 from the imaging preparation position 301 or the imaging position 302 so as to match the attention position 10a with the surgical operation position.

The medical staff (surgeon, assistant, etc.) registers the surgery-performing tabletop position 303 as the registered position in advance by using the registration operation reception part 75 (85) of the operating device 7 (8). The medical staff (surgeon, assistant, etc.) sets the surgery-performing tabletop position 303 which is the registered position as a movement destination of the tabletop 1 by using the registration operation reception part 75 (85) of the operating device 7 (8), in a state where the tabletop 1 is located at the imaging preparation position 301 or the imaging position 302. While the move operation reception part 73 (83) receives the move operation (the instruction to move) with the surgery-performing tabletop position 303 being set as the movement destination of the tabletop 1, the articulated robot arm 3 to move the tabletop 1 so as to match the attention position 10a with the surgical operation position.

Finally, the tabletop 1 is moved by the articulated robot arm 3 to the surgery-performing tabletop position 303 where the attention position 10a (the operative field position, the tumor position) of the patient 10 on the tabletop 1 coincides with the surgical operation position observable by the surgical microscope 700.

### Second Embodiment

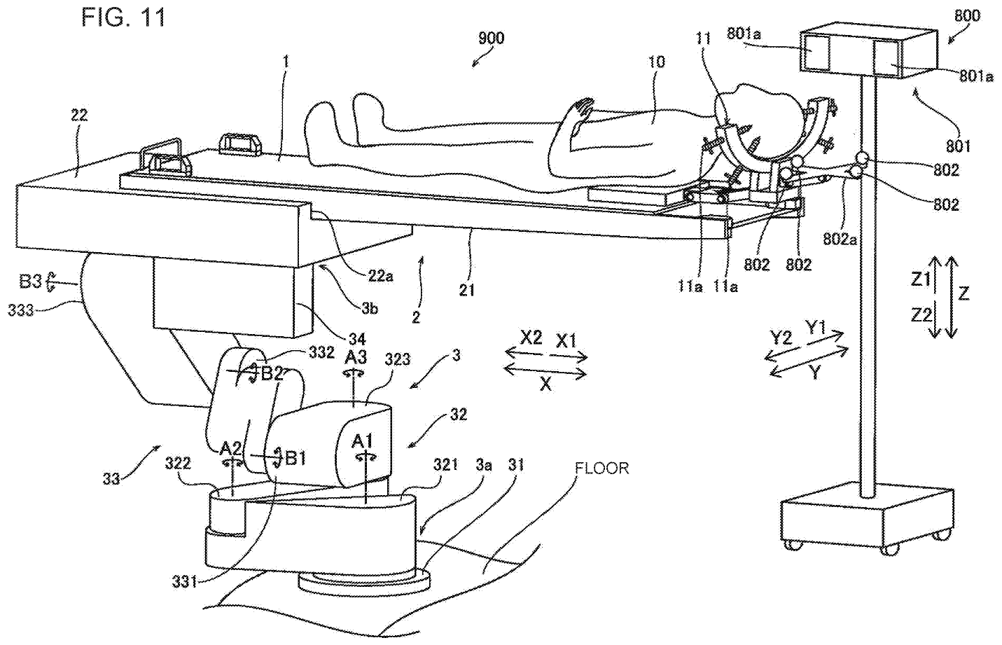

Next, a second embodiment is described with reference to FIGS. 11 to 13. In a second embodiment, unlike a first embodiment described above, the attention position information is obtained by using a surgical navigation system. The configurations of a second embodiment same as those of a first embodiment are designated by the same reference numerals in the drawings, and the description thereof may be omitted for avoid redundancy.

In a second embodiment, as illustrated in FIG. 11, a surgical navigation system 800 is provided. The surgical navigation system 800 includes a spatial position detecting device 801, and a position detection marker 802. The spatial position detecting device 801 is provided as an optical tracking system. The spatial position detecting device 801 includes a plurality (two) of cameras 801a for position detection. Each of the cameras 801a may be an infrared camera capable of detecting infrared rays, for example. The spatial position detecting device 801 is configured to obtain position information indicating a spatial position based on the detection results of the position detection marker 802 by the plurality of cameras 801a.

The position detection marker 802 includes a plurality (four) of position detection markers 802. The plurality of position detection markers 802 may be configured as a passive type or an active type. In the case of the passive type, each of the position detection markers 802 may be a reflective marker having a reflective surface that reflects a detection light from the spatial position detecting device 801 (such as infrared rays). In this case, the spatial position detecting device 801 detects the reflected lights (detection lights) from the position detection markers 802. In the case of the active type, each of the position detection markers 802 may be an LED marker having an LED that emits a detection light from the LED. In this case, the spatial position detecting device 801 detects the detection lights emitted from the LEDs of the position detection markers 802. Each of the position detection markers 802 is formed in a spherical shape. The plurality of the position detection markers 802 are supported by a support portion 802a. The support portion 802a supports the plurality of the position detection markers 802 at different positions from each other (positions separated from each other). For example, the support portion 802a is formed in an X-shape and supports the position detection markers 802 at four tips of the X-shape, respectively.

In a second embodiment, the position detection markers 802 are disposed on the patient 10 or in the vicinity of the patient 10. Thus, it is possible to easily obtain the position information of the patient 10, by means of the position detection markers 802 disposed on the patient or in the vicinity of the patient 10. Specifically, the position detection markers 802 are disposed on the head holder 11. Thus, it is possible to easily obtain the position information of the head of the patient 10, by means of the position detection markers 802 disposed on the head holder 11.

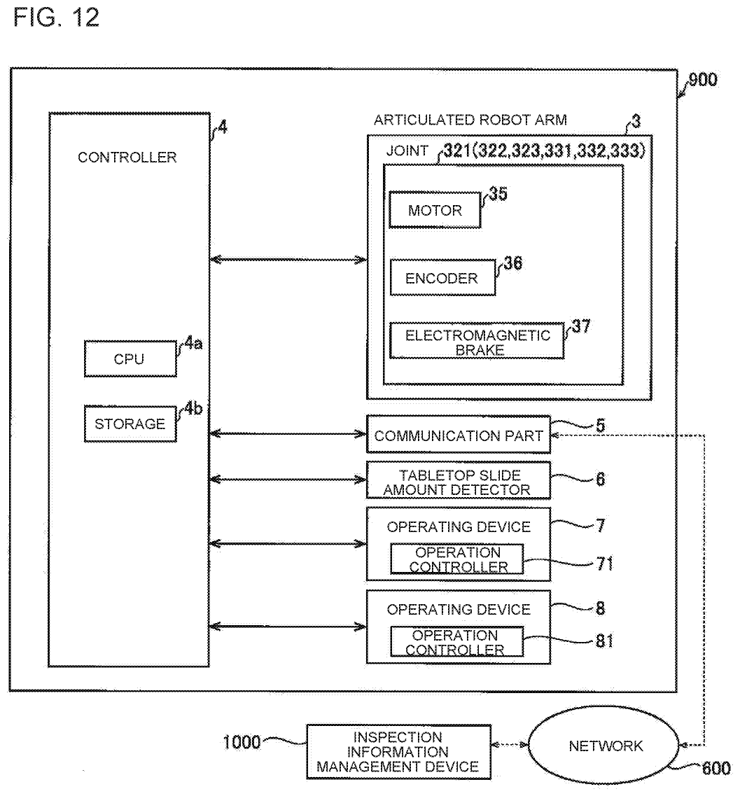

In a second embodiment, as illustrated in FIGS. 11 and 12, an inspection information management device 1000 (see FIG. 12) is configured to acquire the attention position information indicating the attention position 10a of the patient 10 (see FIGS. 10A to 10C), based on the captured image of the patient 10 captured by the medical imaging device 400 (see FIG. 7) and the position information of the position detection markers 802 detected by the spatial position detecting device 801 for detecting the position of the patient 10 placed on the tabletop 1. Thus, by utilizing the spatial position detecting device 801 and the position detection markers 802, it is possible to easily and accurately obtain the attention position information.

Specifically, based on the inspection information management device 1000 is configured to obtain the attention position information indicating the attention position 10a (see FIGS. 10A to 10C) of the patient 10 in the operating room 200, based on the three-dimensional position information of the head of the patient 10 in the operating room 200 (see FIG. 7) detected by the spatial position detecting device 801 and the image of the patient 10 captured by the medical imaging device 400 (see FIG. 7).

Then, as in a first embodiment, the communication part 5 of the robotic operating table 900 is configured to receive the attention position information from the inspection information management device 1000. Further, as in a first embodiment, the articulated robot arm 3 of the robotic operating table 900 is configured to rotate the tabletop 1 about the attention position 10a based on the attention position information received from the inspection information management device 1000. Further, as in a first embodiment, the articulated robot arm 3 of the robotic operating table 900 is configured, based on the attention position information received from the inspection information management device 1000, to move the tabletop 1 to match the attention position 10a with the surgical operation position at which the medical staff performs the surgery while observing the patient 10 by the surgical microscope 700 (see FIG. 7) disposed in the operating room 200 (see FIG. 7). Further, the controller 4 of the robotic operating table 900 is configured to determine the attention position 10a of the patient 10 on the tabletop 1, based on the attention position information received from the inspection information management device 1000.

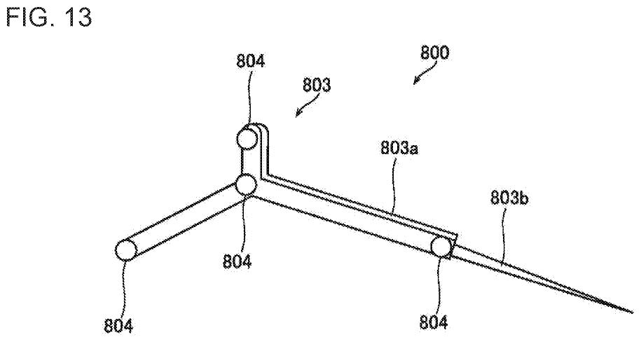

In a second embodiment, as illustrated in FIG. 13, the surgical navigation system 800 includes a pointer 803. A plurality (four) of position recognition marks 804 are attached to the pointer 803. The pointer 803 includes a support portion 803a and a pointer body 803b. The support portion 803a supports the plurality of the position recognition markers 804 at different positions from each other (positions separated from each other). The pointer body 803b is formed in a shape that tapers gradually toward a distal end thereof. In the surgical navigation system 800, the spatial position detecting device 801 (see FIG. 11) is configured to recognize the distal end position and the pointing direction of the pointer body 803b of the pointer 803, based on the detection results of the plurality of the position recognition markers 804. With this configuration, it is possible to confirm the approach angle and the approach direction to the attention position 10a through a monitor (not illustrated) during the surgery.

In a second embodiment, the medical staff can specify an arbitrary position in the operating room 200, by using the pointer 803. In a case where an arbitrary position in the operating room 200 is specified by means of the pointer 803, the communication part 5 of the robotic operating table 900 receives specified position information indicating the arbitrary position specified by the pointer 803. Further, the articulated robot arm 3 of the robotic operating table 900 is configured, based on the specified position information received, to move the tabletop 1 to the arbitrary position specified by the pointer 803. With this configuration, after the medical staff simply uses the pointer 803 to specify an arbitrary position to be treated, the tabletop 1 is moved to the specified arbitrary position. Thus, it is not necessary to register a position where the medical staff want to treat or surgery as a registered position in advance. Therefore, it is possible to reduce the surgical operation time and the labor of the medical staff.

### (Modifications)

It should be understood that embodiments disclosed herein are illustrated by way of example in every respect and not limit the invention. The scope of the invention is indicated by the claims rather than the explanation of the above one or more embodiments and also embraces all changes that come within the meaning and range of equivalents of the claims.

For example, in first and second embodiments described above, the medical imaging device is the MRI device. However, the invention is not limited thereto. In one or more embodiments, the medical imaging device may be a CT (computed tomography) device.

Also, in first and second embodiments described above, the attention position is the operative field position or the tumor position. However, the invention is not limited to this. In one or more embodiments, the attention position may be a position other than the operative field position or the tumor position as long as the attention position is a position specified based on the image of the patient captured by the medical imaging device.

Also, in first and second embodiments described above, the surgical operation position is the position to execute surgery while observing the tumor of the head of the patient with the medical microscope. However, the invention is not limited to this. In one or more embodiments, the surgical operation position may be a position to execute surgery while observing an affected part (for example, a tumor) in a part of the patient other than the head with the medical microscope. In one or more embodiments, the surgical operation position may be a position to execute surgery while visually (directly) observing an affected part (for example, a tumor) in the patient.

Also, in first and second embodiments described above, the robotic operating table includes the tabletop slide mechanism. However, the invention is not limited to this. In one or more embodiments, the robotic operating table may not necessarily include the tabletop slide mechanism. In this case, the second end of the robot arm may directly support the tabletop.

In first and second embodiments described above, the tabletop is provided with the head holder. However, the invention is not limited to this. In one or more embodiments, the tabletop may not necessarily be provided with the head holder in a case where the robotic operating table is used for surgery other than neurosurgery or the like, for example.

Also, in first and second embodiments described above, the robotic operating table is provided with the two operating devices. However, the invention is not limited to this. In one or more embodiments, the robotic operating table may be provided with one operation device or provided with three or more operating devices.

In first and second embodiments described above, the operating devices are connected to the controller so as to be capable of making wired communication with the controller. However, the invention is not limited to this. For example, the operation device may be wirelessly communicable with the controller.

Also, in first and second embodiments described above, the horizontal articulated assembly includes the three horizontal joints. However, the invention is not limited to this. In one or more embodiments, the horizontal articulated assembly may include two horizontal joints or include four or more horizontal joints.

Also, in first and second embodiments described above, the vertical articulated assembly includes the three vertical joints. However, the invention is not limited to this. In one or more embodiments, the vertical articulated assembly may include two vertical joints or include four or more vertical joints.

Also, in first and second embodiments described above, the articulated robot arm is provided with a series of the three horizontal joints and a series of the three vertical joints. The invention is not limited to this. In one or more embodiments, a vertical articulated robot with one or more portions in each of which joints adjacent to each other have their rotation axes orthogonal to each other, may be used as the articulated robot arm.

Also, in first and second embodiments described above, the articulated robot arm has seven degrees of freedom. However, the invention is not limited to this. In one or more embodiments, the articulated robot arm may have six or fewer degrees of freedom or have eight or more degrees of freedom. However, it is preferable for the robot arm to have six or more degrees of freedom.

Also, in first and second embodiments described above, the controller is provided in the base. However, the invention is not limited to this. In one or more embodiments, a control box having a casing in which the controller is housed may be provided. For example, the control box may be disposed at any position in the operating room or the control box may be disposed in a control center near the operating room.

Also, in a second embodiments described above, the position detection makers are disposed in the vicinity of the patient (on the head holder). However, the invention is not limited to this. In one or more embodiments, the position detection marker(s) may be disposed on the patient. In a case where the position detection marker(s) is disposed in the vicinity of the patient, the position detection marker(s) may be provided at a position(s) other than the head holder.

In a second embodiment described above, a marker(s) for MRI may be arranged in the vicinity of the tumor in the brain of the patient when the image of the patient is captured by the MRI device. In this case, it may be possible to more accurately grasp the three-dimensional position of the tumor in the patient image by using the pointer.

In a second embodiment described above, the spatial position detecting device is the optical type spatial position detecting device. However, the invention is not limited to this. In one or more embodiments, the spatial position detecting device may be an image recognition type, a magnetic type, a mechanical type, or the like other than the optical type.

In the disclosure, the surgical operation position may be defined as a specific position set in advance with respect to the coordinate system of the operating room or the coordinate system of the robotic operating table. The specific position may be set to a field of view of the medical microscope provided in the operating room. The specific position may be set to the position where a distal end(s) of a medical treatment tool(s) (a surgical instrument(s)) provided in the operating room is(are) to be operated, in a case where the surgery is executed without the medical microscope.

The invention includes other embodiments in addition to the above-described embodiments and modifications without departing from the spirit of the invention. The embodiments and modifications are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

1. A robotic operating table communicable with an inspection information management device via a network, comprising:
a tabletop on which a patient can be placed;
an articulated robot arm that supports the tabletop to be movable; and
a communication part to communicate with the inspection information management device, wherein
the communication part is configured to receive, from the inspection information management device, attention position information indicating an attention position in the patient specified based on an image of the patient captured by a medical imaging device, and
the articulated robot arm is configured, based on the attention position information received from the inspection information management device, to move the tabletop to a surgery-performing tabletop position which is a position of the tabletop at the time when surgery is executed on the patient.

2. The robotic operating table according to claim 1, wherein
the surgery-performing tabletop position is the position of the tabletop at which the attention position matches with a surgical operation position to execute surgery on the patient.

3. The robotic operating table according to claim 2, wherein
the surgical operation position is a position to execute surgery while observing a tumor in a head of the patient with a medical microscope,
the attention position is a position of the tumor in the patient, and
the attention position information is tumor position information indicating the position of the tumor.

4. The robotic operating table according to claim 1, wherein
the articulated robot arm is configured to move the tabletop to an imaging position which is a position of the tabletop at which the image of the patient on the tabletop is captured with the medical imaging device, and the surgery-performing tabletop position.

5. The robotic operating table according to claim 1, wherein
one of the robotic operating table and the inspection information management device is configured to determine the attention position in the patient with respect to the tabletop, based on the attention position information and image-capturing tabletop position information indicating a position of the tabletop at the time when the image of the patient is captured by the medical imaging device.

6. The robotic operating table according to claim 5, wherein
the one of the robotic operating table and the inspection information management device is configured to determine the attention position in the patient on the tabletop at the time when surgery is executed on the patient, based on on-tabletop attention position information indicating the attention position in the patient with respect to the tabletop, and surgery-performing tabletop position information indicating the position of the tabletop at the time when surgery is executed on the patient.

7. The robotic operating table according to claim 1, further comprising:
a tabletop holder that holds the tabletop to be slidable; and
a tabletop slide mechanism configured to slide the tabletop with respect to the tabletop holder, wherein
the articulated robot arm is configured to support the tabletop via the tabletop holder.

8. The robotic operating table according to claim 7, wherein
the medical imaging device comprises a magnetic resonance imaging (MRI) device, and
the robotic operating table is configured to control the articulated robot arm to move the tabletop holder to an imaging preparation position for the MRI device, and then control the tabletop slide mechanism to slide the tabletop to an imaging position at which the image of the patient is captured by the MRI device.

9. The robotic operating table according to claim 1, wherein
the inspection information management device is configured to obtain the attention position information, based on position information of a position detection marker for detecting a position of the patient on the tabletop detected by a spatial position detecting device and the captured patient image.

10. The robotic operating table according to claim 9, wherein
the position detection marker is disposed on the patient or in the vicinity of the patient.

11. The robotic operating table according to claim 9, wherein
the tabletop is provided with a head holder to hold a head of the patient, and
the position detection marker is disposed on the head holder.

12. A robotic operating table communicable with an inspection information management device via a network, comprising:
a tabletop on which a patient can be placed;
an articulated robot arm that supports the tabletop to be movable; and
a communication part to communicate with the inspection information management device, wherein
the communication part is configured to receive, from the inspection information management device, attention position information indicating an attention position in the patient specified based on an image of the patient captured by a medical imaging device, and
the articulated robot arm is configured to rotate the tabletop about the attention position in the patient on the tabletop based on the attention position information received from the inspection information management device.

13. The robotic operating table according to claim 12, wherein
the attention position is a position of a tumor of the patient, and
the attention position information is tumor position information indicating the position of the tumor.

14. The robotic operating table according to claim 12, wherein
the articulated robot arm is configured to move the tabletop so as to match the attention position with a surgical operation position to execute surgery on the patient, based on the attention position information received from the inspection information management device.

15. The robotic operating table according to claim 12, wherein
the articulated robot arm is configured to move the tabletop to an imaging position which is a position of the tabletop at which the image of the patient is captured by the medical imaging device, and a surgery-performing tabletop position which is a position of the tabletop at the time when surgery is executed on the patient.

16. The robotic operating table according to claim 14, wherein
the surgical operation position is a position to execute surgery while observing a tumor of a head of the patient with a medical microscope.

17. The robotic operating table according to claim 12, wherein
one of the robotic operating table and the inspection information management device is configured to determine the attention position in the patient with respect to the tabletop, based on the attention position information and image-capturing tabletop position information indicating a position of the tabletop at the time when the image of the patient is captured by the medical imaging device.

18. The robotic operating table according to claim 17, wherein
the one of the robotic operating table and the inspection information management device is configured to determine the attention position in the patient on the tabletop at the time when surgery is executed on the patient, based on on-tabletop attention position information indicating the attention position in the patient with respect to the tabletop, and surgery-performing tabletop position information indicating the position of the tabletop at the time when surgery is executed on the patient.

19. The robotic operating table according to claim 12, wherein
the inspection information management device is configured to obtain the attention position information, based on position information of a position detection marker for detecting a position of the patient on the tabletop detected by a spatial position detecting device and the captured patient image.

20. The robotic operating table according to claim 19, wherein
the position detection marker is disposed on the patient or in the vicinity of the patient.

21. The robotic operating table according to claim 19, wherein
the tabletop is provided with a head holder to hold a head of the patient, and
the position detection marker is disposed on the head holder.
